(19) United States
(12) Patent Application Publication
(10) Pub. No.: US 2018/0000676 A1
(43) Pub. Date: Jan. 4, 2018

(54) Surgical Extremity Position Supporting Device

(71) Applicants: Dustin D. Poole, Princeton, TX (US); Thomas Stillings, Plano, TX (US)

(72) Inventors: Dustin D. Poole, Princeton, TX (US); Thomas Stillings, Plano, TX (US)

(21) Appl. No.: 15/607,785

(22) Filed: May 30, 2017

Related U.S. Application Data
(60) Provisional application No. 62/343,046, filed on May 30, 2016.

(51) Int. Cl. A61G 13/12; A61G 13/10; A61F 5/37

(52) U.S. Cl. CPC A61G 13/1205; A61F 5/3769; A61G 13/102

(57) ABSTRACT

Disclosed is a disposable sterile device for supporting the position of an extremity during surgery. The device comprises a base and a support panel. The support panel is for receiving a limb of a patient thereon. The top surface of the support panel is transversely concave whereby, upon the reception of the limb thereon, the contour of the limb substantially matches with that of the upper surface of the support panel. The support panel is disposed in operative communication with the base whereby, the support panel is, with respect to the base, adapted to be longitudinally adjustably inclined and elevated. The support panel is further adapted to be transversely tiltable.

### BACKGROUND

The invention disclosed herein generally relates to the technical field of surgical devices. More particularly, the invention disclosed herein relates to a surgical device for supporting the positioning of the upper and lower extremities of a patient during a surgical procedure.

Medical professionals, especially surgeons are constantly faced with the challenge of addressing clinical issues concerning the upper or lower extremities of the human body. Extremities are frequently exposed to impact by external objects and stress due to repetitive usage, which may cause serious ankle impairment or damage to limbs. These types of injuries frequently require surgeries and similar treatment options to rectify the problem. The surgical equipment utilized to treat injuries to the upper or lower extremities often overlooks the issue of positioning the injured extremity in a secure position during surgery or treatment. Medical equipment that allows the doctor or surgeon to access the patient's limb in order to ensure optimal quality of medical care is often inadequate. Typically, medical personnel use bulky and heavy devices for surgical support that require sterilization, increased time and effort for assembling and transport. This consumes more time and expense for both the patient and medical staff and can reduce the effectiveness of the treatment. Surgical positioning devices, which are compact, require less time and effort to set up are thus required.

Conventionally, surgical positioning equipment does not have the capability of providing the angle and tilt that is required to perfectly position the arm or leg to present the injury in an optimum position for the surgeon. Moreover, equipment having such capabilities can be large, cumbersome, and have to be cleaned and sterilized prior to use. This takes considerable time and effort to assemble the group of items for support. In a surgical environment, reducing patient preparation time is advantageous. Alternatively, even after careful adjustment, the angle or tilt may be off by a little, which is enough to make the operation difficult and affecting the treatment. A surgical positioning apparatus for supporting the extremities, which can provide the inclination and tilt, is required.

Hence, there is a long felt but unresolved need for a surgical device, which is compact and requires less time and effort to set up. Moreover, there is a need for such a device, which is portable, disposable, and inexpensive. Furthermore, there is a need for such a device, which can provide easy adjustment to angle and tilt.

### SUMMARY

An embodiment of the present invention comprises a surgical extremity position supporting device, which comprises a support panel, a base and a connector assembly releasingly connecting the support panel and the base. The connector assembly connects the support panel and the base such that, the support panel is adapted to be longitudinally adjustably inclined with respect to the base at different angles. The connector assembly also enables the support panel to be transversely tiltable.

The support panel is adapted to receive a limb (be it a hand or a leg) of a patient on the top surface thereof. The support panel comprises a substantially rectangular structure extending between a pair of opposingly-disposed longitudinal edges and a pair of opposingly-disposed transverse edges. The support panel is transversely concave so as to, again, provide better comfort as the contour of the limb substantially matches with that of the concave top surface of the support panel. The concave shape also helps in holding the limb in place. The support panel comprises two pairs of opposingly-disposed strap receptacles disposed on the longitudinal edges thereof. Each pair of strap receptacles is adapted to run a strap therethrough wherein, the strap is intended for securing the limb to the support panel.

The support panel further comprises a plurality of parallel, spaced-apart, elongate, transverse grooves extending between the longitudinal edges. The grooves are intended for collecting fluids from the limb and from the surgery. A hinge member descends from the bottom surface of the support panel. More particularly, the hinge member is disposed closer to one of the lateral edges of the support panel while being central thereto. The support panel further comprises a plurality of parallel, spaced-apart, elongate, transverse top indentations disposed on the bottom surface thereof wherein, the each top indentation extends between the longitudinal edges.

The base comprises a rectangular structure defined between a pair of opposingly-disposed longitudinal and transverse sides. The top surface of the base comprises a plurality of parallel, spaced-apart, elongate, transverse bottom indentations wherein, each bottom indentation extends between the longitudinal sides. A hinge receptacle is disposed on the top surface thereof. More particularly, the hinge receptacle is disposed closer to one of the lateral sides while being laterally centrally aligned. The hinge receptacle is adapted to removably engage the hinge member therewithin at which point, a hinge joint is formed between the support panel and the base. The hinge joint also complies to allow the lateral angular tilt of the support panel thereabout. The base further comprises a reservoir disposed therewithin. The reservoir is intended to collect and store fluids from the grooves. More particularly, when the support panel is transversely tilted, the fluids collected within the grooves are drained onto the top surface of the base. Now, the top surface of the base comprises multiple, elongate reservoir openings disposed thereon. The fluids are received within the reservoir via the reservoir openings.

The connector assembly further comprises a planar connector, which is adapted to engage the bottom surface of the support panel and the top surface of the base so as to maintain a desired incline between the support panel and the base. The connector comprises a planar top member, a planar bottom member and a hinge pin hingedly connecting the top and bottom members at which point, the top member oscillates about the bottom member. The bottom member comprises an elongate bottom base and a stem integrally extending perpendicularly from the bottom base centrally. The stem comprises a central opening and an extremity opening.

The top member comprises a slightly curved, elongate top base and a semi-circular projection integrally extending downwardly from the centre of the top base. The top member further comprises a semi-annular member, the extremities of which integrally connect that of the top base so as to form a substantially semi-circular member. When the top and bottom members are hingedly secured to one another, the extremity opening straddles the semi-circular projection, while the semi-annular member is freely received within the central opening. A hinge pin is received through the extremity of the stem and the semi-circular projection thereby forming the hinged connection between the top and bottom members. In order to engage the connector between the support panel and the base, the top base of the top member is snugly received within a top indentation while the bottom base of the bottom member is snugly received within the bottom indention. Once the connector engages the support panel and the base, owing to the hinged connection between the top and bottom members, the support panel is rendered tiltable about the base.

Other features and advantages will become apparent from the following description of the preferred embodiments, taken in conjunction with the accompanying drawings.

### BRIEF DESCRIPTION OF THE DRAWINGS

Various embodiments of the disclosed system and method are described herein with reference to the accompanying drawings, which form a part of this disclosure, wherein:

FIG. 1, according to an embodiment of the present invention, is an illustration of a top perspective view of the device.

FIG. 2, according to an embodiment of the present invention, is an illustration of a bottom perspective view of the device.

FIG. 3, according to an embodiment of the present invention, is an illustration of a side view of the device.

FIG. 4, according to an embodiment of the present invention, is an illustration of an exploded side view of the device.

FIG. 5, according to an embodiment of the present invention, is an illustration of a perspective view of the support panel.

FIG. 6, according to an embodiment of the present invention, is an illustration of a top view of the support panel.

FIG. 7, according to an embodiment of the present invention, is an illustration of a bottom view of the support panel.

FIG. 8, according to an embodiment of the present invention, is an illustration of a longitudinal side view of the support panel.

FIG. 9, according to an embodiment of the present invention, is an illustration of a transverse side view of the support panel.

FIG. 10, according to an embodiment of the present invention, is an illustration of a perspective view of the base.

FIG. 11, according to an embodiment of the present invention, is an illustration of a top view of the base.

FIG. 12, according to an embodiment of the present invention, is an illustration of a bottom view of the base.

FIG. 13, according to an embodiment of the present invention, is an illustration of a side view of the base.

FIG. 14, according to an embodiment of the present invention, is an illustration of a front view of the connector.

FIG. 15, according to an embodiment of the present invention, is an illustration of a side view of the connector.

FIGS. 16A through 16D, according to an embodiment of the present invention, are sequential illustrations of the inclination of the support panel being raised.

FIGS. 17A and 17B, according to an additional embodiment of the present invention, are sequential illustrations of the support panel being elevated.

FIG. 18, according to an alternate embodiment of the present invention, is an illustration of a perspective view of the device.

FIG. 19, according to an alternate embodiment of the present invention, is an illustration of a top perspective view of the panel assembly.

FIG. 20, according to an alternate embodiment of the present invention, is an illustration of a bottom perspective view of the panel assembly.

FIG. 21, according to an alternate embodiment of the present invention, is an illustration of a perspective view of the base.

FIG. 22, according to an alternate embodiment of the present invention, is an illustration of a top view of the base.

FIG. 23, according to an alternate embodiment of the present invention, is an illustration of a perspective view of the connector assembly.

FIG. 24, according to an alternate embodiment of the present invention, is an illustration of a side view of the connector assembly.

FIG. 25, according to an alternate embodiment of the present invention, is an illustration of a perspective view of the support member.

FIGS. 26A through 26C, according to an alternate embodiment of the present invention, are sequential illustrations of the panel assembly being raised.

FIGS. 27A through 27C, according to an alternate embodiment of the present invention, are sequential illustrations of the panel assembly being inclined.

FIGS. 28A through 28C, according to an alternate embodiment of the present invention, are sequential illustrations of the panel assembly being tilted.

The various aspects of the present disclosure mentioned above are described in further detail with reference to the aforementioned figures and the following detailed description of exemplary embodiments.

### FIGURES—REFERENCE NUMERALS

10—Extremity Position Supporting Device
12—Support Panel
14—Base
16—Connector
18—Groove
20—Strap Receptacle
22—Strap
24—Top Indentation
26—Hinge Member
28—Bottom Indentation
30—Reservoir Opening
32—Hinge Receptacle
34—Suction Hole
36—Gripping Leg
38—Reservoir
40—Bottom Member
42—Top Member
44—Hinge Pin
46—Panel Assembly
48—Additional Panel
50—Panel Connector
52—Connector Assembly
54—Base Rod
56—Support Member
58—Longitudinal Top Rod
60—Transverse Bottom Rod
62—Tab
64—Bottom Strip Member
66—Top Strip Member
68—Inclination Knob
70—Tilt Knob
72—Base Wall

### DETAILED DESCRIPTION

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

Embodiments of the present invention are directed to a sterile disposable surgical device for comfortably supporting the position of an extremity (i.e., a limb) of a human patient during a surgery. The device, in doing so as aforementioned, also provides a 360° viewpoint for the doctor(s) or surgeon(s) involved in the surgical procedure whereby, the progress of said surgical procedure is not hampered. The surgical device is disposable. The device is preferably made of medical-grade polypropylene plastic, which is very durable, provides compliance during use, is highly resistant to hospital and cleaning chemicals, and is typically used in this type of application. The device also helps in user-controlled integrated soft tissue retraction.

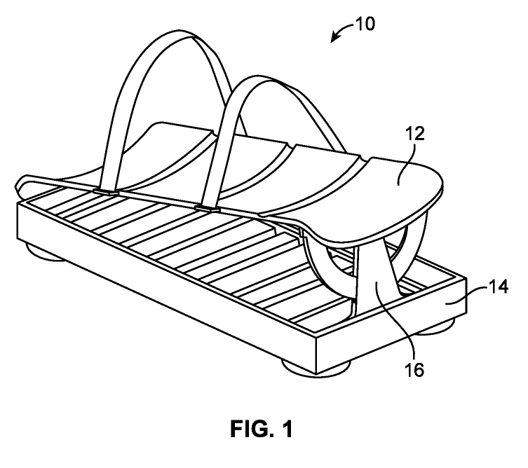

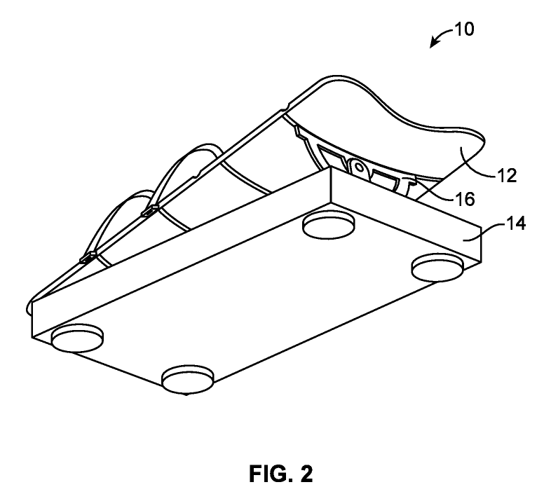

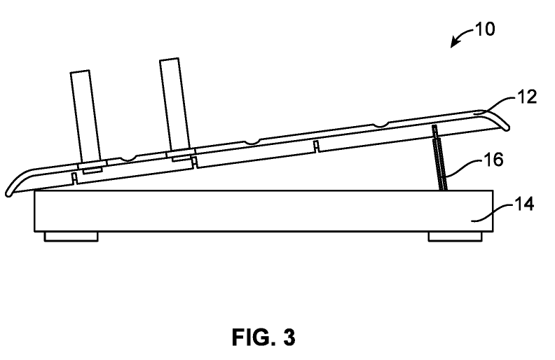

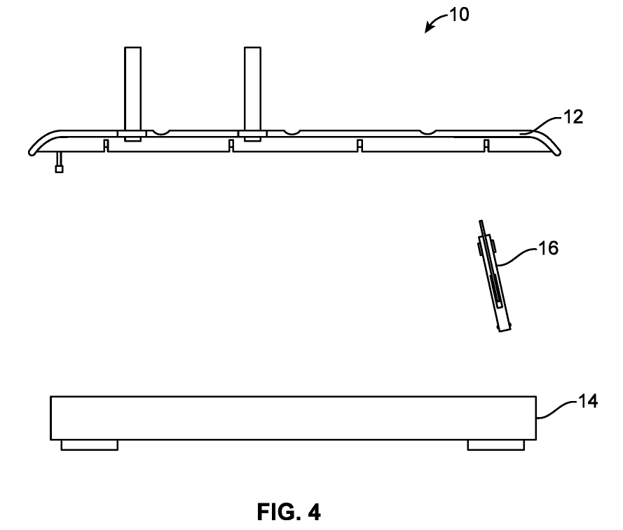

Referring to FIGS. 1 through 4, in a preferred embodiment, the device 10, which is simple in construction, comprises a support panel 12, a base 14 and a connector assembly releasingly connecting the support panel 12 and the base 14. The connector assembly connects the support panel 12 and the base 14 such that, the support panel 12 is adapted to be longitudinally inclined with respect to the base 14 at different angles. The connector assembly also enables the support panel 12 to be transversely tiltable.

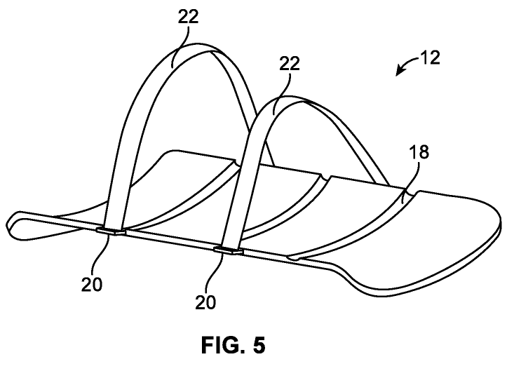

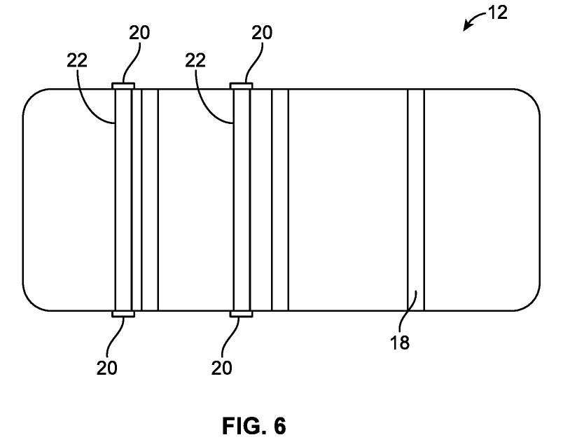

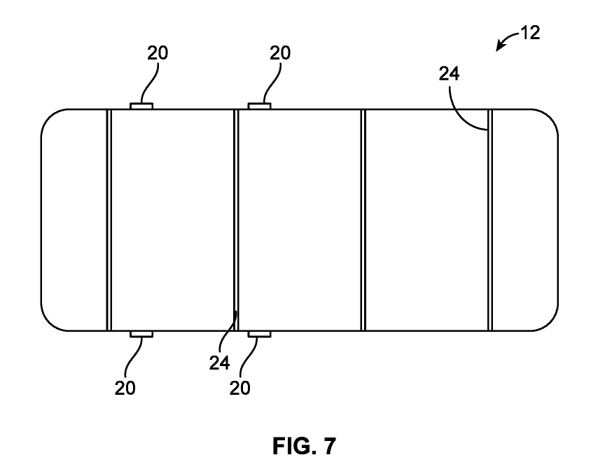

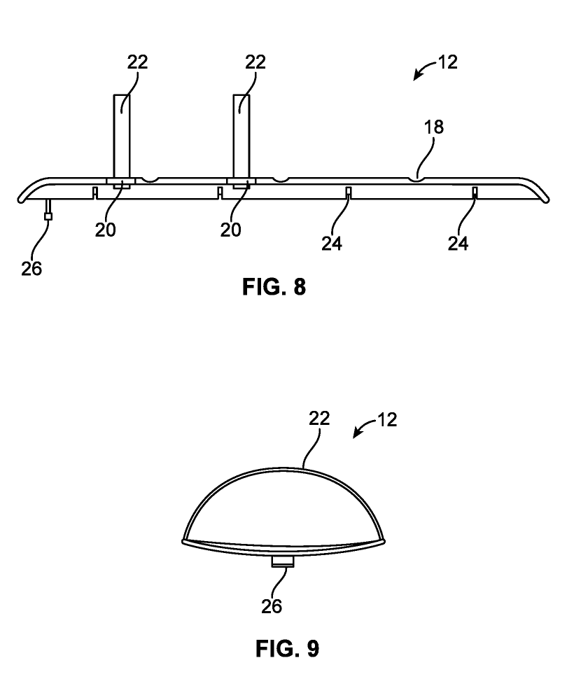

Referring to FIGS. 5 through 9, the support panel 12 is adapted to receive a limb (be it a hand or a leg) of a patient on the top surface thereof. The support panel 12 comprises a substantially rectangular structure extending between a pair of opposingly-disposed longitudinal edges and a pair of opposingly-disposed transverse edges wherein, the length of the longitudinal edge is greater than that of the transverse edge. As can be easily appreciated from FIG. 8, both the transverse edge portions of the support panel 12 are curved downwardly to provide better comfort for the received limb. The support panel 12 is transversely concave so as to, again, provide better comfort as the contour of the limb substantially matches with that of the concave top surface of the support panel 12. The concave shape also helps in holding the limb in place. The support panel 12 comprises a means for securing the limb thereon. More particularly, the securing means comprises two pairs of opposingly-disposed strap receptacles 20 disposed on the opposingly-faced longitudinal edges of the support panel 12. The securing means further comprises straps 22, each of which pertaining to a strap receptacle 20 pair. Once the limb is received on the support panel 12, the straps 22 are looped through the strap receptacles 20 so as to secure the limb to the support panel 12.

Still referring to FIGS. 5 through 9, the support panel 12 further comprises a plurality of parallel, spaced-apart, elongate, transverse grooves 18 extending between the longitudinal edges. The grooves 18 are intended for collecting fluids from the limb and from the surgery. Notably, each groove 18 is of uniform, substantially semi-circular cross-section and is perpendicular to the longitudinal edges. A hinge member 26 descends from the bottom surface of the support panel 12. More particularly, the hinge member 26 is disposed closer to one of the lateral edges of the support panel 12 while being central thereto. The support panel 12 further comprises a plurality of parallel, spaced-apart, elongate, transverse top indentations 24 disposed on the bottom surface of the support panel 12 wherein, the each top indentation 24 extends between the longitudinal edges such that, each top indentation is 24 is perpendicular to the longitudinal edges. Notably, each top indentation 24 is of a uniform, substantially rectangular U-shaped cross-section. The utility of the hinge member 26 and the top indentations 24 will become apparent from the following body of text.

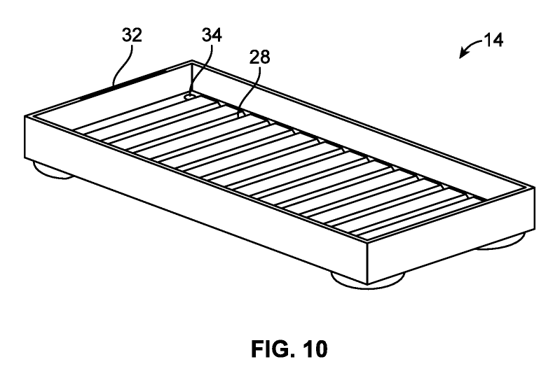

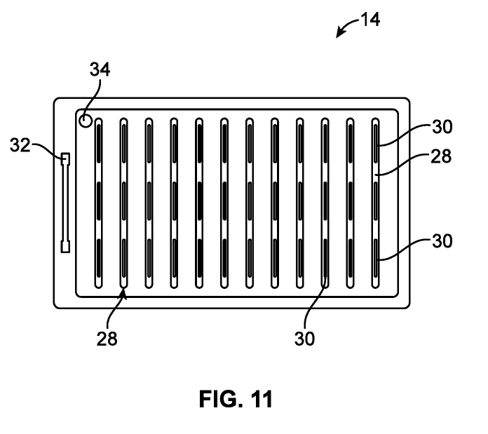

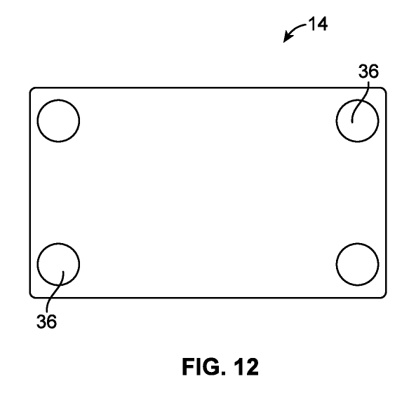

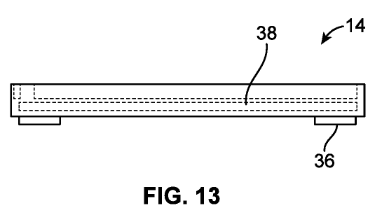

Referring to FIGS. 10 through 13, the base 14 comprises a rectangular structure extending between a pair of opposingly-disposed longitudinal sides and a pair of opposingly-disposed transverse sides wherein, the length of the longitudinal side is greater than that of the transverse side. The top surface of the base 14 comprises a plurality of parallel, spaced-apart, elongate, transverse bottom indentations 28 wherein, each bottom indentation 28 extends between the longitudinal sides such that, each bottom indentation 28 is perpendicular to the longitudinal sides. Notably, each bottom indentation 28 is of a uniform, substantially rectangular U-shaped cross-section. A hinge receptacle 32 is disposed on the top surface thereof. More particularly, the hinge receptacle 32 is disposed closer to one of the lateral sides while being laterally centrally aligned. The hinge receptacle 32 is adapted to removably engage the hinge member 26 therewithin at which point, a hinge joint is formed between the support panel 12 and the base 14. The hinge joint also complies to allow the lateral angular tilt of the support panel 12 thereabout wherein, the utility of such said movement will become apparent from the following body of text. Notably, the hinge member 26 and the hinge receptacle 32 are part of the connector assembly.

Sill referring to FIGS. 10 through 13, the base 14 further comprises a reservoir 38 disposed therewithin. The reservoir 38 is intended to collect and store fluids from the grooves 18. More particularly, when the support panel 12 is transversely tilted, the fluids collected within the grooves 18 are drained onto the top surface of the base 14. Now, the top surface of the base 14 comprises multiple, elongate reservoir openings 30 aligned within the bottom indentations 28. The fluids are received within the reservoir 38 via the reservoir openings 30. The top surface of the base 14 further comprises a suction hole 34 disposed thereon wherein, the suction hole 34 is in fluid communication with the reservoir 38. The suction hole 34 is intended for discarding therethrough the fluids collected within the reservoir 38. Preferably, a suction tool is employed to vacuum-pump the fluids out of the reservoir 38. The bottom surface of the base 14 is attached with four gripping legs 36 disposed at the four corners thereof. Each leg 36 is made of a non-slippery material, preferably a rubbery one, so as to hold onto the surface whereon the device 10 is placed.

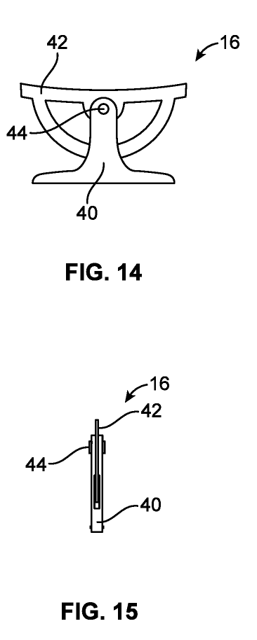

Referring to FIGS. 14 and 15, the connector assembly further comprises a planar connector 16, which is adapted to releasably engage the bottom surface of the support panel 12 and the top surface of the base 14 so as to maintain a desired incline between the support panel 12 and the base 14. The connector 16 comprises a planar top member 42, a planar bottom member 40 and a hinge pin 44 hingedly connecting the top and bottom members 40 and 42 at which point, the top member 42 oscillates about the bottom member 40. The bottom member 40 comprises an elongate bottom base and a stem integrally extending perpendicularly from the bottom base centrally. As can be appreciated from FIG. 15, the stem comprises a central opening and an extremity opening, the utility of both of which will become apparent from the following body of text.

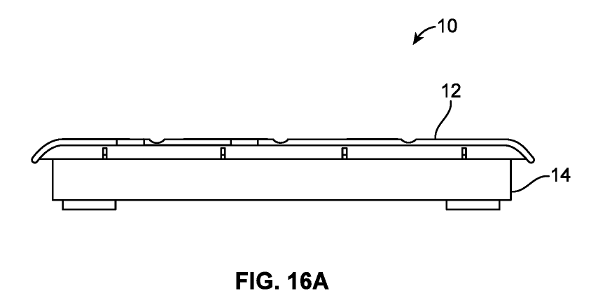

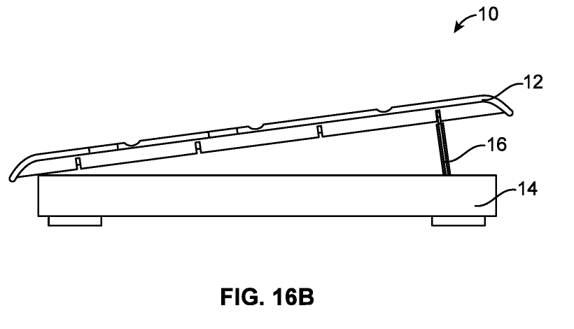

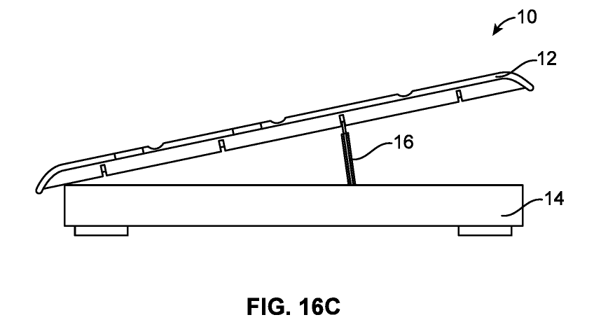

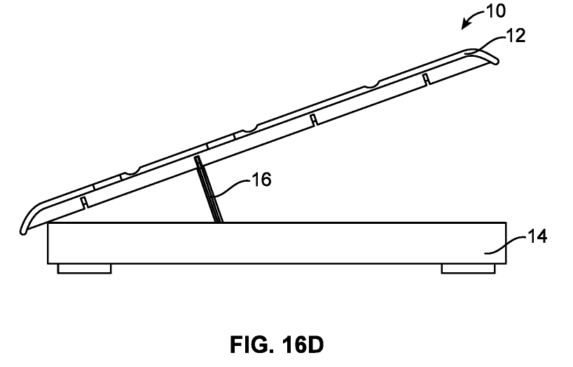

Still referring to FIGS. 14 and 15, the top member 42 comprises a slightly curved, elongate top base and a semi-circular projection integrally extending downwardly from the centre of the top base. The top member 42 further comprises a semi-annular member, the extremities of which integrally connect that of the top base so as to form a substantially semi-circular member as shown in FIG. 14. When the top and bottom members 40 and 42 are hingedly secured to one another, the extremity opening straddles the semi-circular projection, while the semi-annular member is received within the central opening. A hinge pin 44 is received through the extremity of the stem and the semi-circular projection thereby forming the hinged connection between the top and bottom members 40 and 42. In order to engage the connector 16 between the support panel 12 and the base 14, the top base of the top member 42 is snugly received within a top indentation 24 while the bottom base of the bottom member 40 is snugly received within the bottom indention 28. Once the connector 16 engages the support panel 12 and the base 14, owing to the hinged connection between the top and bottom members 40 and 42, the support panel 12 is rendered tiltable about the base 14. As can be appreciated from FIGS. 16A through 16D, as the connector 16 is moved from one longitudinal extremity towards the other longitudinal extremity (of the hinge joint), the angle of inclination between the support panel 12 and the base 14 increases.

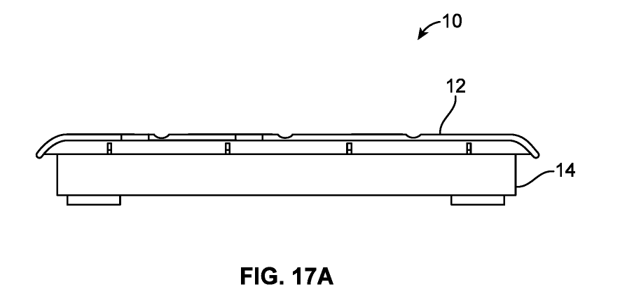

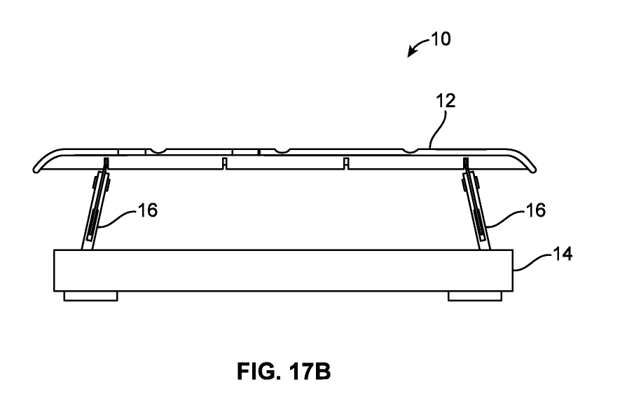

Referring to FIGS. 17A and 17B, in an additional embodiment, an additional connector 16 is provided wherein, the two connectors are releasably engaged at the two longitudinal ends of the support panel 12 and the base 14. This enables the support panel 12 to be elevated above the base 14. Notably, the support panel 12 is tiltable even in this structural configuration. In one embodiment, the support panel 12 is adapted to be interchangeable with another support panel of a different dimensional configuration.

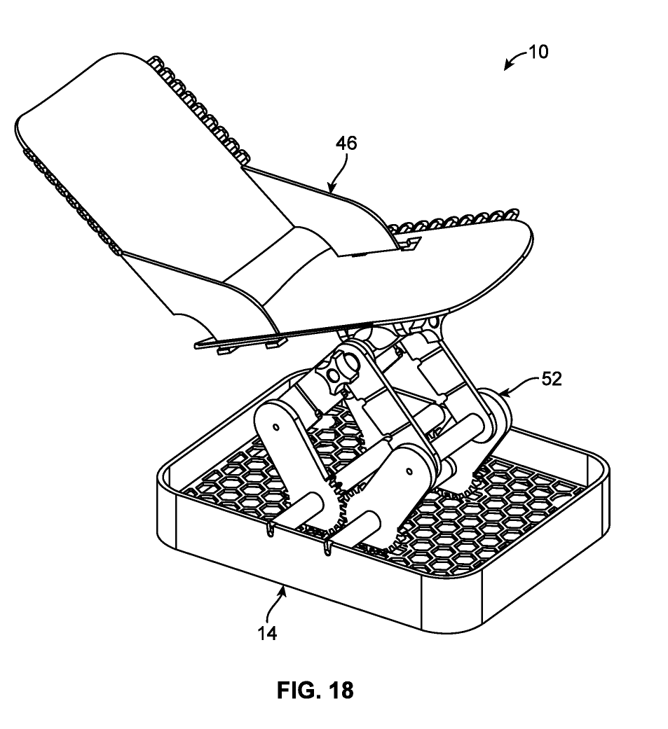

Referring to FIGS. 18 through 20, in an alternate embodiment, the device 10 comprises a panel assembly 46, a base 14 and a connector assembly 52 connecting the panel assembly 46 and the base 14. The panel assembly 46 comprises a support panel 12, an additional panel 48, and a panel connector 50 connecting the support panel 12 and the additional panel 48. In one embodiment, the support panel 12, additional panel 48, and the panel connector 50 are detachable from one another. The support panel 12 is adapted to receive a limb (be it a hand or a leg) of a patient on the top surface thereof. The support panel 12 comprises a substantially rectangular structure extending between a pair of opposingly-disposed longitudinal edges and a pair of opposingly-disposed transverse edges wherein, the length of the longitudinal edge is greater than that of the transverse edge. The support panel 12 is transversely concave so as to provide better comfort as the contour of the limb substantially matches with that of the concave top surface of the support panel 12. The concave shape also helps in holding the limb in place. The support panel 12 comprises a means for securing the limb thereon. More particularly, the securing means comprises two pairs of opposingly-disposed strap receptacles 20 disposed on the opposingly-faced longitudinal edges of the support panel 12. The securing means further comprises straps 22, each of which pertaining to a strap receptacle 20 pair. Once the limb is received on the support panel 12, the straps 22 are looped through the strap receptacles 20 so as to secure the limb to the support panel 12.

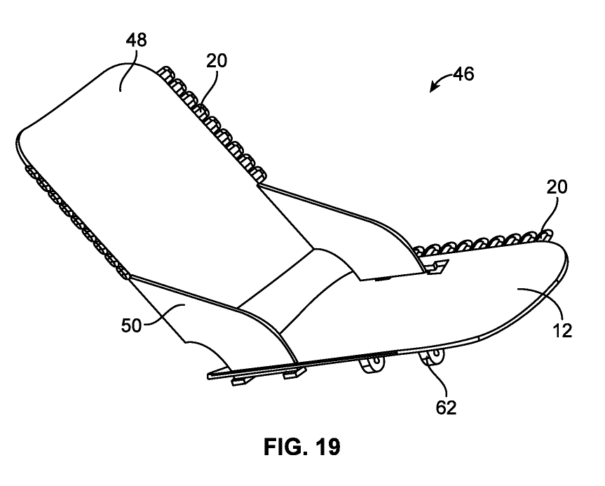

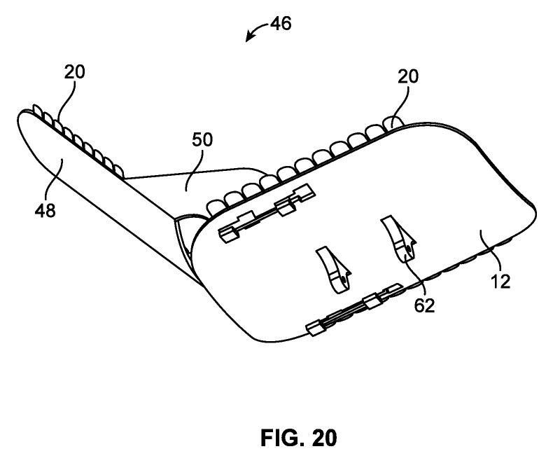

Referring to FIGS. 19 and 20, the additional panel 48 is also similar to the support panel 12 as it is transversely concave and comprises a plurality of pairs of strap receptacles 20 extending from the longitudinal edges thereof. A planar panel connector 50 connects the support and the additional panels 12 and 48 such that, the support and the additional panels 12 and 48 are inclined with respect to each other. More particularly, the support and the additional panels 12 and 48 are substantially perpendicular to one another. In one embodiment, the inclination between the support and the additional panels 12 and 48 is adjustable. The location of the connection of panel connector 50 to the support and the additional panels 12 and 48 is closer to the respective longitudinal edges thereof so as not to occupy the space of both the support and the additional panels 12 and 48. A pair of spaced-apart tabs 62 descends from bottom surface of the support panel 12 centrally. Each tab 62 comprises a thorough hole extending centrally therethrough. The utility of the pair of tabs 62 will become apparent from the following body of text.

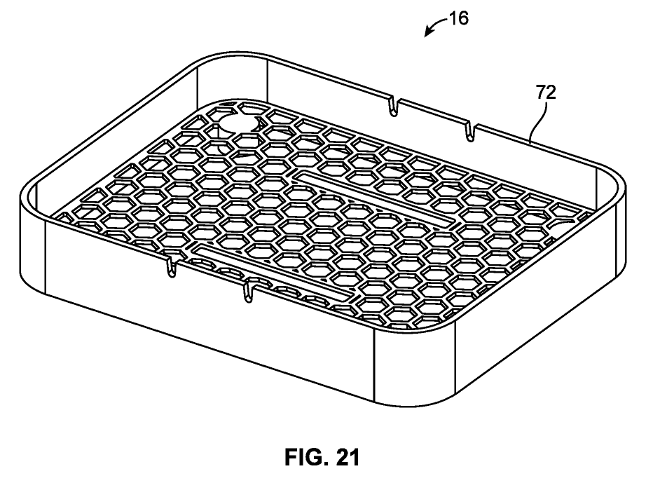

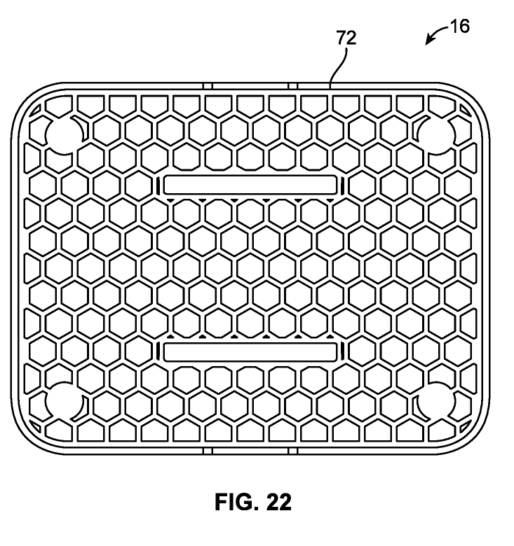

Referring to FIGS. 21 and 22, the base 14 comprises a rectangular structure extending between a pair of opposingly-disposed longitudinal walls and a pair of opposingly-disposed transverse walls. Notably, the opposingly-disposed longitudinal walls will hereinafter be referred to as base walls 72. The base 14 further comprises a reservoir (not shown) disposed therewithin. The reservoir is intended to collect and store fluids emanating from the limb and expended during the surgical procedure performed on the limb secured to the panel assembly 46. More particularly, when the support panel 12 is transversely or longitudinally tilted (the matter of how will be explained in the following body of text), the fluids collected on the support panel 12 are drained onto the top surface of the base 14. Now, the top surface of the base 14 comprises multiple reservoir openings (not shown) through which, the fluids are received within the reservoir. The top surface of the base 14 further comprises a suction hole (not shown) disposed thereon wherein, the suction hole is in fluid communication with the reservoir. The suction hole is intended for discarding therethrough the fluids collected within the reservoir. Preferably, a suction tool is employed to vacuum-pump the fluids out of the reservoir. The bottom surface of the base 14 is attached with four gripping legs (not shown) disposed at the four corners thereof. Each leg is made of a non-slippery material, preferably a rubbery one, so as to hold onto the surface whereon the device 10 is placed.

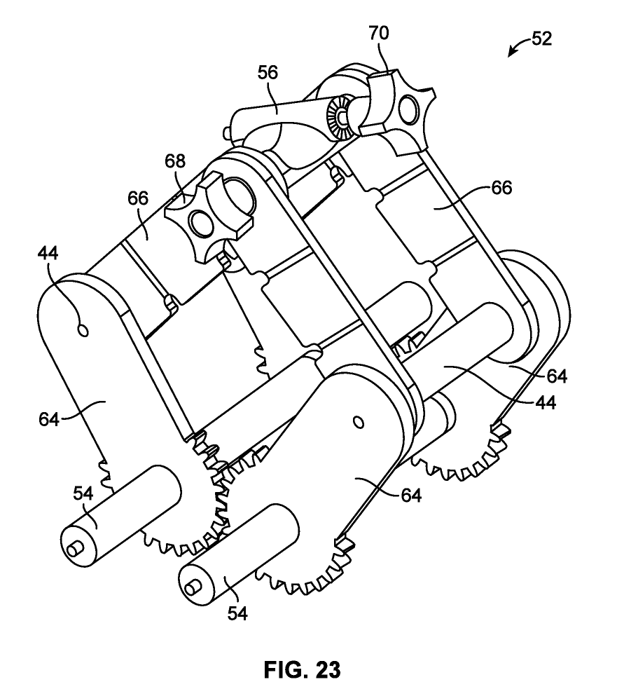

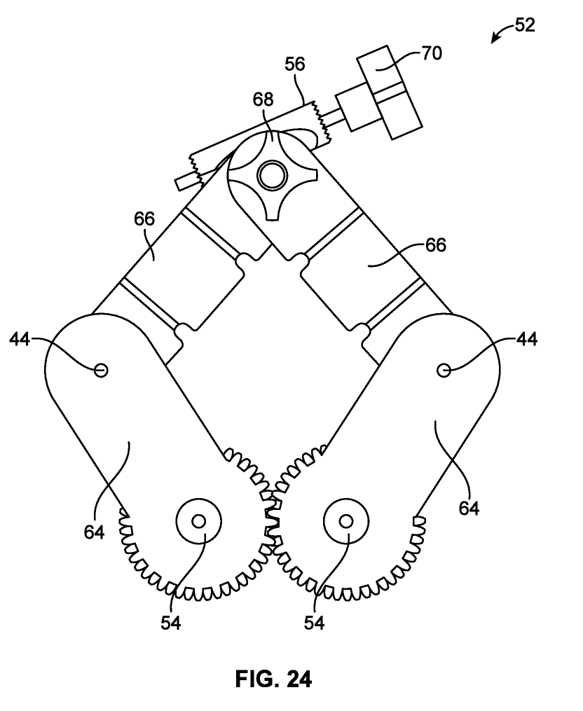

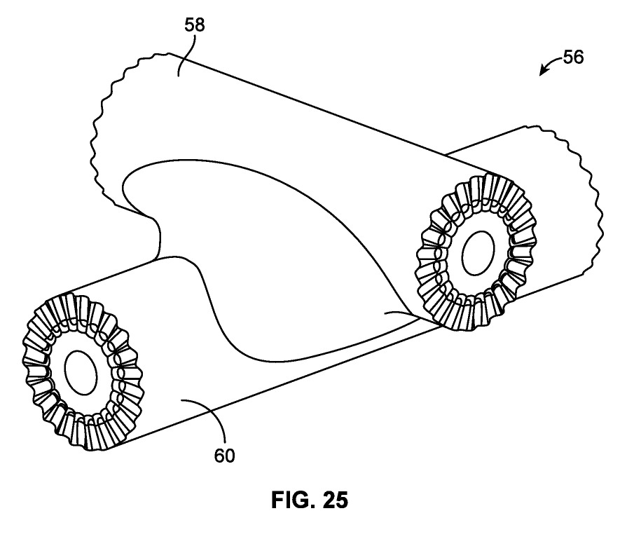

Referring to FIGS. 23 and 24, the connector assembly 52 comprises a pair of parallel, spaced-apart base rods 54 extending between a pair of opposingly-disposed base walls 72 such that, the base rods 54 are perpendicular to the base walls 72. The connector assembly 52 further comprises two pairs of interconnected hinge assembles and a support member 56 to which the support panel 12 is secured. A hinge assembly pair comprises a pair of elongate, planar, bottom strip members 64 extending between a proximal and a distal extremity. The proximal extremity of each bottom strip member 64 is rotably coupled to a base rod 54. Further, the proximal extremity of each bottom strip member 64 comprises a gear. The gears of the pair of bottom strip members 64 (rotably coupled to the base rods 54) are meshed so as to inhibit free rotary movement thereof with respect to the base rods 54. A hinge assembly pair further comprises a pair of elongate, planar, top strip members 66, which too extend between a proximal and a distal extremity. As enabled by an elongate hinge pin 44, the proximal extremity of each top strip member 66 is hingedly coupled to a distal extremity of a bottom strip member 64.

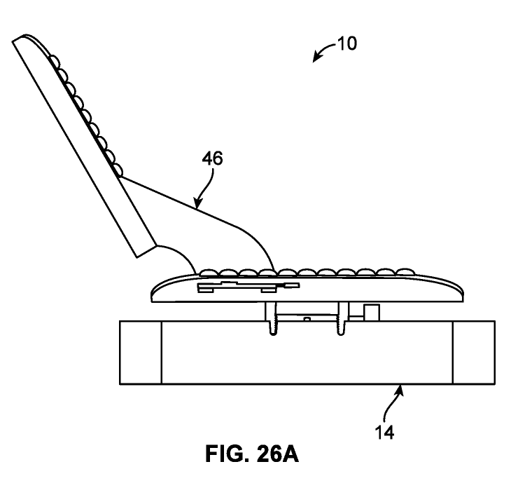

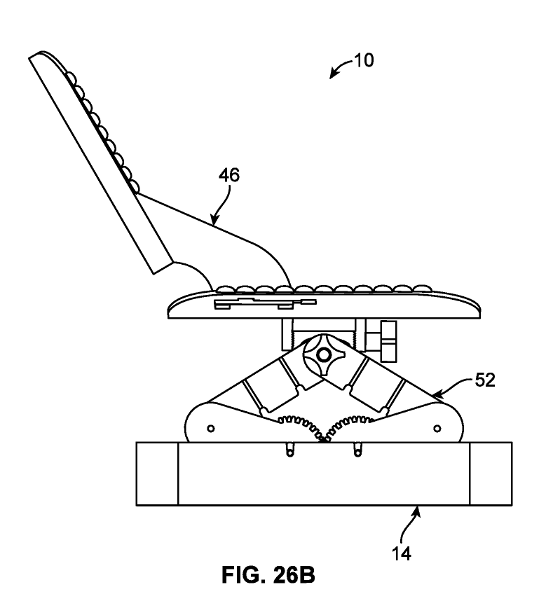

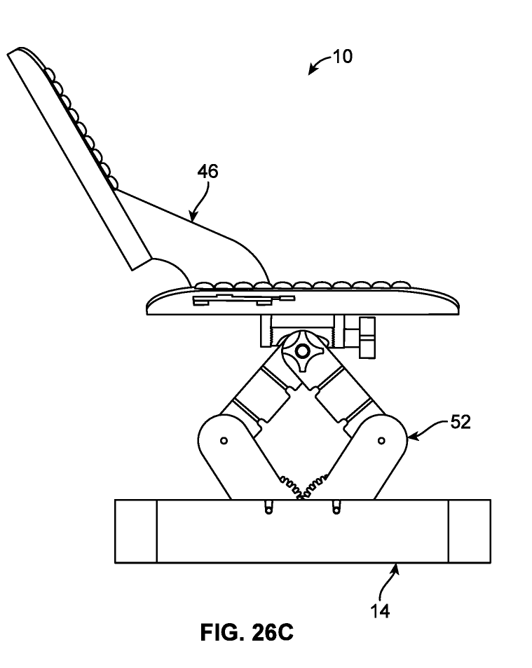

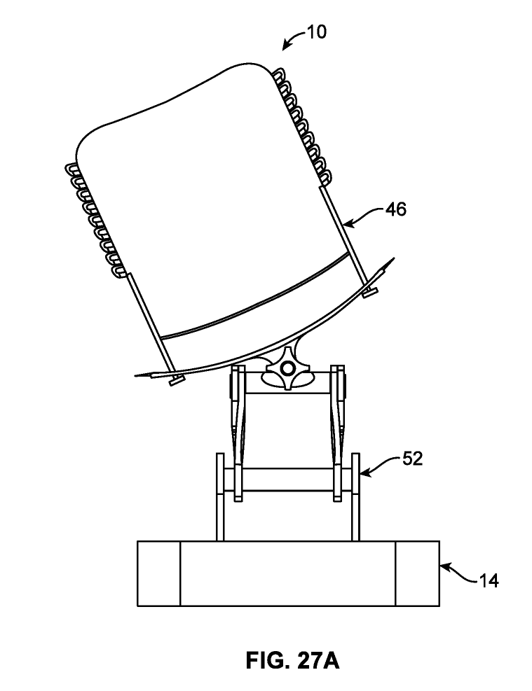

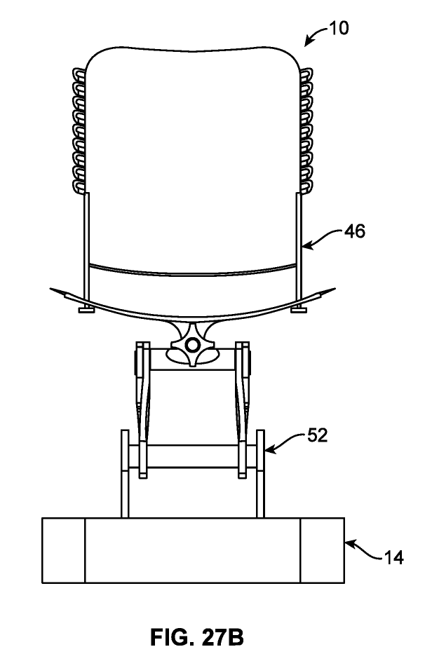

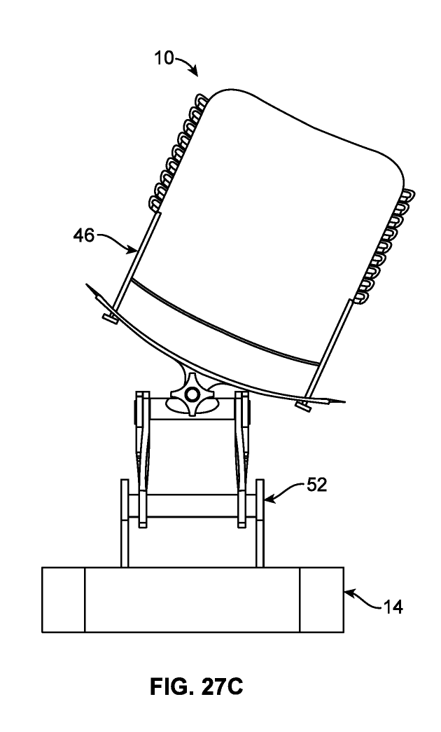

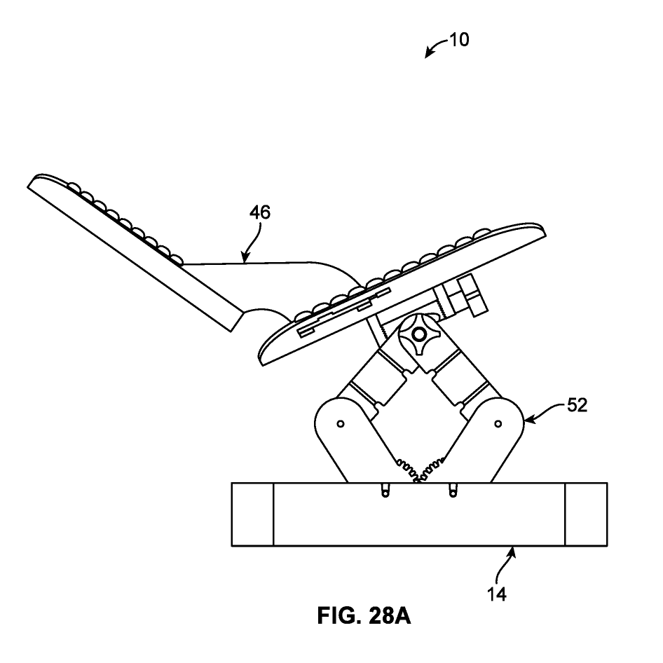

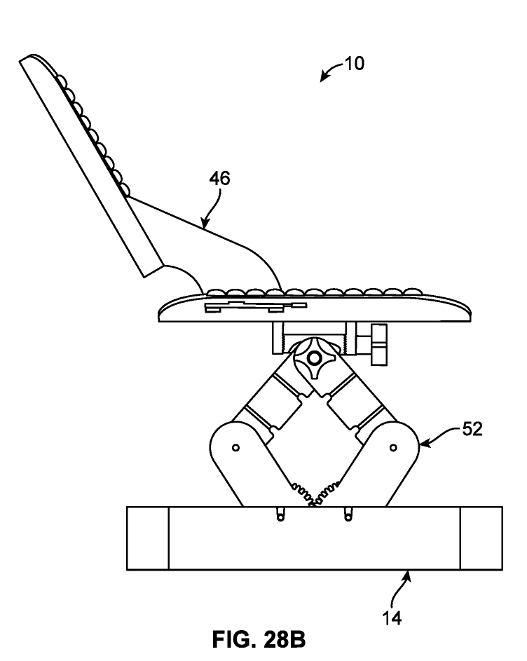

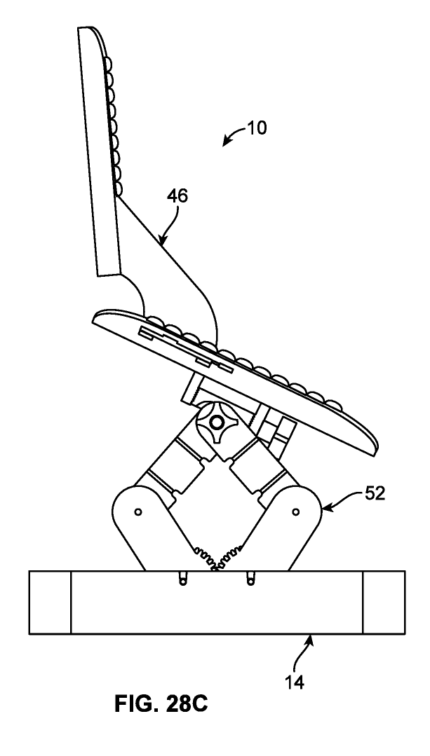

Still referring to FIGS. 23 and 24, the connector assembly 52 further comprises a support member 56 comprising a longitudinal top rod 58 and a transverse bottom rod 60 wherein, the top and bottom rods 58 and 60 are perpendicular to one another. More particularly, the top and bottom rods 58 and 60 form a plus-shaped structure as viewed from top. The top rod 58 is removably rotably fitted between the pair of tabs 62 whereby, the panel assembly 46 is transversely adjustably tiltable about the top rod 58 as shown in FIGS. 27A through 27C. The connector assembly 52 further comprises a tilt knob 70, which when tightened locks the tilt position of the panel assembly 46. Notably, the tilt knob 70 is axial to the top rod 58. Each extremity of the bottom rod 60 is rotably coupled to the distal extremities of the pair of top strip members 66. This enables panel assembly 46 to be longitudinally adjustably inclined about the base 14 as facilitated by the bottom rod 60 as shown in FIGS. 28A through 28C. The connector assembly 52 further comprises an inclination knob 68, which when tightened locks the inclination position of the panel assembly 46. Notably, the inclination knob 68 is axial to the bottom rod 60. Notably, a hinge pin 44 commonly facilitates two hinged connections between the top and bottom strip members 64 and 66. As can be appreciated from FIGS. 26A through 26C, the connector assembly 52 also enables the panel assembly 46 to be elevated and lowered about the base 14. In one embodiment, the support panel 12 is adapted to be interchangeable with another support panel of a different dimensional configuration.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A sterile surgical extremity position supporting device comprising a base and a support panel for receiving a limb of a patient thereon, the top surface of the support panel being transversely concave whereby, upon the reception of the limb thereon, the contour of the limb substantially matches with that of the upper surface of the support panel, the support panel disposed in operative communication with the base whereby, the support panel is, with respect to the base, adapted to be longitudinally adjustably inclined, adjustably elevated or both adjustably elevated and inclined; the support panel further adapted to be transversely tiltable.

2. The device of claim 1 wherein, the top surface of the support panel comprises a plurality of parallel transverse grooves for collecting fluid, upon the transverse tilt of the support panel, the collected fluid from the grooves is in turn collected within a reservoir disposed within the base; the reservoir accessible via a plurality of reservoir openings disposed on the top surface of the base.

3. The device of claim 2 wherein, the top surface of the base comprises a suction hole for discarding the fluid collected within the receptacle therethrough.

4. The device of claim 1 wherein, the transverse edge portions of the support panel are downwardly curved.

5. The device of claim 1 further comprising an additional panel removably extending substantially perpendicularly from the support panel; the additional panel disposed closer to a longitudinal extremity of the support panel.

6. The device of claim 1 wherein, the support panel further comprises a means for securing the limb thereto.

7. The device of claim 1 wherein, the means for securing comprises a plurality of pairs of opposingly-disposed strap receptacles and a plurality of transverse adjustable straps for securing the limb thereto; each strap looping through a pair of strap receptacles.

8. The device of claim 1 further comprising a connector assembly connecting the base and the support panel for facilitating the adjustable inclination therebetween, the connector assembly comprising:
(a) a hinge joint comprising:
(i) a hinge member extending downwardly from a longitudinal extremity of bottom surface of the support panel; and
(ii) a hinge receptacle disposed at an extremity of the top surface of the base, the hinge receptacle adapted to releasably receive the hinge member therewithin thereby forming the hinge joint, which facilitates the angular displacement of the support panel about the hinge joint, the hinge joint configured to be compliant to the transverse tiltable motion of the support panel; and
(b) a planar connector comprising:
(i) a planar top member, the top extremity of which is adapted to releasably engage the bottom surface of the support panel; and
(ii) a planar bottom member, the bottom extremity of which is adapted to releasably engage the top surface of the base;
the top and bottom members being hingedly secured to one another whereby, upon the connector engaging the base and the support panel, the support panel is tiltable about the base.

9. The device of claim 8 wherein, the top surface of the base and the bottom surface of support panel comprises a plurality of parallel and transverse top and bottom indentations respectively disposed thereon, the top and bottom indentations for receiving the top and bottom ends of the connector respectively; the incline between the base and support panel increases as the position of the connector is moved from one extremity towards the extremity that is closer to the hinge joint.

10. The device of claim 8 wherein, the connector assembly further comprises an additional planar connector wherein, in order to elevate the support panel above the base, the additional connector is secured at one end, while the other connector is secured at the other opposite end; the hinge member disengaged from the hinge receptacle in the event of the support panel being elevated.

11. The device of claim 1 further comprising a connector assembly connecting the base and the support panel for adjustably elevating and lowering the support panel with respect to the base, longitudinally adjustably inclining the support panel with respect to the base and transversely adjustably tilting the support panel with respect to the base, the connector assembly comprising:
(a) a pair of spaced-apart, parallel base rods extending substantially centrally between the inner surfaces of opposingly-faced walls of the base;
(b) at least two pairs of hinge assemblies; and
(c) a support member comprising:
(i) a longitudinal top rod rotably releasably secured to the bottom of the support panel such that, the support panel is transversely adjustably tiltable thereabout; and
(ii) a transverse bottom rod connected to the pair of base rods by means of the at least two pairs of hinge assemblies whereby, the support member, and thereby the support panel, is upwardly extendible and downwardly retractable about the pair of base rods, the bottom rod being parallel to the pair of base rods, the bottom rod further enabling longitudinal adjustable inclination of the support panel with respect to the base.

12. The device of claim 11 wherein, the support member is disposed in operative communication with an inclination knob such that, when the inclination knob is tightened, the longitudinal inclination position of the support panel is locked; the inclination knob being axial to the bottom rod.

13. The device of claim 11 wherein, the support member is disposed in operative communication with a tilt knob such that, when the tilt knob is tightened, the transverse tilt position of the support panel is locked; the tilt knob being axial to the top rod.

14. The device of claim 11 wherein, each hinge assembly comprises:
(a) a pair of elongate, planar bottom strip members, each of which extending between a proximal and distal extremity, each bottom strip member rotably connected to base rod at the proximal extremity thereof; and
(b) a pair of elongate, planar top strip members, each of which extending between a proximal and distal extremity, the proximal extremity of each top strip member hingedly connected to distal extremity of a bottom strip member rod, the distal extremity of each top strip member is rotably connected to an extremity of bottom rod.

15. The device of claim 14 wherein, the proximal extremity of each bottom strip member comprises a gear; the gears of a pair of bottom strip members being meshed so as to inhibit free angular movement thereof.

16. The device of claim 14 wherein, the at least two pairs of hinge assemblies comprise two pair of opposingly-disposed hinge assemblies.

17. The device of claim 16 wherein, the hinge joints between the top and bottom strip members pertaining to the two pairs of hinge assemblies are connected by a common, elongate hinge pin.

18. The device of claim 11 wherein, the top and bottom rods, as viewed from top, form a plus-shaped structure, each extremity of the top rod is rotably connected to a tab descending from the bottom surface of the support panel thereby facilitating the transverse tilt of the support panel.

19. The device of claim 1 wherein, the bottom surface of the base comprises four legs for gripping the surface whereon the base is placed; each leg is placed at a corner of the base.

20. The device of claim 1 being made of medical-grade polypropylene plastic.

21. The device of claim 1 wherein, the support panel is adapted to be interchangeable with another support panel of different dimensional configuration.

22. A sterile surgical extremity position supporting device comprising:
(a) a base comprising a reservoir accessible via a plurality of reservoir openings;
(b) a support panel for receiving a limb of a patient thereon, the top surface of the support panel being transversely concave whereby, upon the reception of the limb thereon, the contour of the limb substantially matches with that of the upper surface of the support panel, the support panel disposed in operative communication with the base whereby, the support panel is, with respect to the base, adapted to be longitudinally adjustably inclined and elevated, the support panel further adapted to be transversely tiltable, the top surface of the support panel comprising a plurality of parallel transverse grooves for collecting fluid, upon the transverse tilt of the support panel, the collected fluid from the grooves is in turn collected within the reservoir, the support panel further comprising a plurality of transverse adjustable straps for securing the limb thereto; and
(c) a connector assembly connecting the base and the support panel, the connector assembly comprising:
(i) a hinge joint comprising:
(1) a hinge member extending downwardly from a longitudinal extremity of bottom surface of the support panel; and
(2) a hinge receptacle disposed at an extremity of the top surface of the base, the hinge receptacle adapted to receive the hinge member therewithin thereby forming the hinge joint, which facilitates the angular displacement of the support panel about the hinge joint, the hinge joint configured to be compliant to the transverse tiltable motion of the support panel; and
(ii) a planar connector comprising:
(1) a planar top member, the top extremity of which is adapted to engage the bottom surface of the support panel; and
(2) a planar bottom member, the bottom extremity of which is adapted to engage the top surface of the base;
the top and bottom members being hingedly secured to one another whereby, upon the connector engaging the base and the support panel, the support panel is tiltable about the base.
